(19) United States
(12) Patent Application Publication
(10) Pub. No.: US 2022/0395438 A1
(43) Pub. Date: Dec. 15, 2022

(54) TOOTH WHITENING COMPOSITION

(71) Applicant: Universitat Autonoma de Barcelona, Bellaterra (ES)

(72) Inventors: Manuel Valiente Malmagro, Sant Cugat del Vallès (ES); Maria Jesús Sánchez Martín, Mollet del Vallès (ES); Clara Babot Marquillas, Barcelona (ES); Jorge Rodríguez Martínez, Villafane (Leon) (ES)

(73) Assignee: Universitat Autonoma de Barcelona, Bellaterra (ES)

(21) Appl. No.: 17/774,301

(22) PCT Filed: Nov. 4, 2020
(86) PCT No.: PCT/EP2020/080878

§ 371 Date: May 4, 2022

(30) Foreign Application Priority Data
Nov. 4, 2019 (EP) 19382958.7

(51) Int. Cl. A61K 8/14; A61K 8/55; A61K 8/23; A61K 8/22; A61K 8/365; A61Q 11/00

(52) U.S. Cl. CPC A61K 8/14; A61K 8/553; A61K 8/23; A61K 8/22; A61K 8/365; A61Q 11/00

(57) ABSTRACT

The present invention relates to a tooth whitening composition, which comprises a reducing agent partially encapsulated in liposomes. It relates also to a method for preparing that tooth whitening composition and to the non-therapeutic cosmetic use of that composition for tooth whitening.

### TECHNICAL FIELD

The present invention belongs to the field of compositions for tooth whitening.

### BACKGROUND ART

The natural colour of teeth is opaque to translucent white or slightly off-white. However, the use of certain foods and tobacco, the process of aging, diseases, trauma, medications, some congenital conditions, and environmental effects can cause teeth to become discoloured. Because whiter teeth are considered to be aesthetically superior to stained or discoloured teeth, there has been a large demand for dental bleaching compositions.

Typical tooth bleaching agents release active oxygen radicals. Such bleaching agents include peroxides, such as hydrogen peroxide, percarbonates and perborates of the alkali and alkaline earth metals, or complex compounds containing hydrogen peroxide, such as carbamide peroxide. Also, peroxide salts of the alkali or alkaline earth metals and peroxyacetic acid are known to be useful in whitening teeth, as disclosed in EP-A-0545594. Peroxyacetic acid may be generated in situ from a precursor comprising labile acyl groups, as disclosed in US-A-2001/0021374. Sodium chlorite is also disclosed as tooth whitening agent, for example, in WO-98/04235.

Current bleaching agents contain both active and inactive ingredients. The active ingredients include usually hydrogen peroxide or carbamide peroxide in a wide range of concentrations (5-35% or 10-35% respectively). However, the major inactive ingredients may include thickening agents, carrier, surfactant and pigment dispersant, preservative, and flavouring.

Patients who have desired to have their teeth whitened have typically done so by applying a bleaching composition to the teeth by means of a dental tray for repeated treatments, or they have had to submit to conventional in-office bleaching techniques that required from 4 to 10 visits to the dental office before clinically significant results were achieved. Clinically significant results are quantifiable by means of colour measurement.

Reducing agents have been used scarcely as tooth whitening agents. In WO-A-95/05148 it is disclosed a composition for reducing or removing surface deposited stains from natural teeth and dental prostheses comprising an effective amount of an orally acceptable sequestering agent and an orally acceptable reducing agent, such as Vitamin C, vitamin E, BHA, BHT, and propyl gallate, in an orally acceptable carrier. In WO-A-01/64175 it is disclosed a “multi-stage” whitening procedure for teeth, wherein different types of whitening agents are used consecutively. One of the agents is papain enzyme activated by additional co-ingredients, such as thiol containing groups, such as cysteine hydrochloride, mercaptoethanol, and dithiotreitol, or metabisulfite salts. In WO-A-99/07818 it is disclosed an ultrasonic water-soluble dental tablet cleansing composition comprising scrubbing particles, a bleaching agent, a bleaching enhancer, a tablet aid, a tablet disintegrator, a tartar control agent, a crystallization inhibitor, a chelating agent, and a pH adjusting agent. As bleaching enhancers are mentioned sodium sulphite, thiosulfate, thiourea and metabisulfite.

Different technical solutions have been disclosed in prior art to improve the tooth whitening effect.

For example, to accelerate the whitening effect of peroxygen compounds, in WO-A-97/02805 it is disclosed the use of a manganese coordination complex compound such as manganese gluconate in combination with the bleaching compound.

Another approach is the formulation of the tooth whitening compound with a gelling compound. In U.S. Pat. No. 5,290,566 it is disclosed a tooth whitening formulation comprising urea peroxide in combination with a gelling agent, which keeps the formulation in contact with the teeth for a period of time sufficient to cause whitening thereof. An anhydrous dental bleaching gel composition comprising propylene glycol, polyethylene glycol, glycerine, neutralized carboxypolymethylene, hydroxypropyl-cellulose, and carbamide peroxide, is disclosed in U.S. Pat. No. 5,718,886. Hydrogen peroxide has also been gelled to improve the bleaching effect by combining it with polyacrylic acid, as disclosed in US-A-2003/0170592. In EP-A-0511782 it is disclosed a sustained-release film-forming polymer composition comprising a specific water-soluble cellulosic polymer; a pharmaceutically or veterinary acceptable oxidising agent; and a pharmaceutically or veterinary acceptable vehicle.

A further approach is the use of an abrasive together with the tooth whitening agent. In EP-A-0535816 it is disclosed an abrasive dentifrice composition containing a peroxide compound, a dicalcium phosphate compound and a metal ion free peroxide compound. Further tooth whitening abrasive compositions are disclosed, for example, in WO-A-97/11675, WO-A-97/21419, and WO-A-99/02126. In CN-A-103356396 it is disclosed a whitening tooth powder, which comprises silicon dioxide, sodium hydrogen carbonate, sodium tripolyphosphate, flavouring agents, sodium metabisulfite and sodium carbonate.

However, in an attempt to increase the efficiency of bleaching agents, such as carbamide peroxide, higher concentrations are used, which leads to the occurrence of most common adverse effects, such as mild to moderate tooth sensitivity and/or gingival irritation, as disclosed in Desai et al., The effect of a chemical activator on tooth bleaching with two different concentrations of carbamide peroxide: An in vitro study, Int. J. Appl. Dental Sci., 2018, 4(1), 286-289.

The use of oxygen-based tooth whitening treatments is associated to controversy over the effects on tooth structure (physical properties of enamel and dentin), such as increased porosity of the superficial enamel structure, demineralization and decreased protein concentration, organic matrix degradation, modification in the calcium: phosphate ratio, calcium loss, reduction of dentin microhardness, and decrease in the flexural strength and flexural modulus of dentin, as disclosed in M. Q. Alqahtani, Tooth-bleaching procedures and their controversial effects: A literature review, The Saudi Dental J., 2014, 26, 33-46.

Despite the numerous proposals available in the state of the art, there is still a need to have new compositions for tooth whitening showing improved efficiency and reduced secondary effects.

### OBJECT OF THE INVENTION

The object of the present invention is a method for preparing a composition for tooth whitening.

It is another aspect of the invention the composition for tooth whitening obtainable according to that method.

It is another aspect of the invention the non-therapeutic cosmetic use of that composition for tooth whitening.

### FIGURES

FIG. 1 discloses the colour change obtained in the in vitro tooth whitening treatments described in Example 3. In ordinates there is the colour change (increase of whiteness) expressed as ΔE for each treatment, wherein treatment 1 represents the negative control (water), treatment 2 comprises aqueous liposomes, treatment 3 comprises sodium metabisulfite, treatment 4 represents 16 wt. % carbamide peroxide, and treatment 5 comprises the composition of the invention according to Example 1, and in abscises there are the different times (3′, 6′, 9′, 14′ and 20′), when the whitening increase was measured. It was observed that the composition of the invention showed a faster and more effective whitening effect than the single components.

FIG. 2 discloses the colour change obtained in in vitro tooth whitening treatments according to international standard ISO 28399 of Example 12. In ordinates there is the colour change (increase of whiteness) expressed as ΔE for each treatment, wherein treatment 1 represents the negative control (water), treatment 2 represents the positive control (citric acid 1 wt. %), treatment 3 represents 16 wt. % carbamide peroxide, treatment 4 represents 35 wt. % hydrogen peroxide, and treatment 5 represents the treatment with the composition disclosed in Example 9. In abscises there are the results for the first, second and third treatment, each at 5-days intervals, and the follow up after one month of the last treatment. It was observed the high efficacy of the tooth whitening treatment with the composition of the invention in comparison to prior art treatments.

FIG. 3 discloses the increase in roughness of the tooth, in ordinates as ΔRa, measured in compliance with ISO 28399, for the treatments disclosed in Example 12, which are in abscises, wherein treatment 1 represents the negative control (water), treatment 2 represents the positive control (citric acid, 1 wt. %), treatment 3 represents 16 wt. % carbamide peroxide, treatment 4 represents 35 wt. % hydrogen peroxide, and treatment 5 represents the treatment with the composition disclosed in Example 9. It was observed that the result for the composition of the invention is substantially similar to that of the positive control, citric acid solution, which is considered safe for dental treatments. ISO 28399 considers tested treatment safe in terms of roughness, if its value is lower than three times the value of the positive control.

### DETAILED DESCRIPTION OF THE INVENTION

The object of the present invention is a method for preparing a tooth whitening composition, which comprises:
1) the step of mixing at least one membrane-forming lipid in an organic solvent in a vessel, and
   either
2a) dispersing the mixture obtained in step 1) in an aqueous solution comprising a reducing agent, and
3a) removing the organic solvent to obtain a composition comprising liposomes comprising the solution of the reducing agent,
   or
2b) removing the organic solvent of the mixture obtained in step 1) to obtain a layer of the membrane-forming lipid on the inner surface of the vessel, and
3b) adding an aqueous solution comprising a reducing agent to the layer of step 2b) to obtain a composition comprising liposomes comprising the solution of the reducing agent.

The authors of the present invention have developed a method for preparing a composition suitable for tooth whitening, which show improved efficiency due to the rapid action, which can lead to reduced secondary effects, such as the weakening of tooth, by means of a reduced time of treatment application. The composition comprises a solution of a reducing agent, partially encapsulated in liposomes. Without being bound to any theory, it is considered that the liposome fraction of the composition remains attached on the surface of the tooth improving the whitening effect of the reducing agent. The composition of the invention shows a synergistic effect in comparison with the use of a reducing agent alone, or liposomes alone.

The term “approximately” or “about” refers to a deviation of plus/minus 10%, preferably plus/minus 5%.

In the present description, as well as in the claims, the singular forms “a”, “an” and “the” include the plural reference unless the context clearly indicates otherwise. The ranges defined by the preposition “between” include also the two ends thereof.

According to the American Dental Association, restoring the natural colour of teeth is called whitening, and whitening teeth beyond their natural colour is called bleaching. However, teeth whitening and teeth bleaching are used interchangeably in prior art. In the present invention a whitening process for teeth covers both the process for restoring the natural colour of teeth by removing stains from the tooth surface and the process for whitening teeth beyond their natural colour.

### Method

In the method of the invention, the at least one membrane-forming lipid is dispersed in an aqueous solution comprising a reducing agent, to obtain a composition comprising liposomes comprising the solution of the reducing agent.

The composition obtained in step 3a) or 3b) of the method comprises:
i. liposomes formed by at least one membrane-forming lipid, which comprise an aqueous solution of the reducing agent, optionally comprising also a buffer system, and
ii. an aqueous solution of the reducing agent, optionally comprising also a buffer system, which is not entrapped in the liposome membrane.

In both cases, a) and b), the solution of the reducing agent stays both inside and outside the membrane-forming lipid.

Liposomes are spontaneously formed when phospholipids are dispersed in aqueous medium, as disclosed in Bangham et al., in Korn, E. D. (Ed.) Methods in Membrane Biology, Vol.1, Plenum Press, New York, 1975, pp. 1-68.

The preparation of liposomes resulting from the dispersion of the at least one membrane-forming lipid in the aqueous solution of a reducing agent, can be carried out by well-known techniques. In general, the preparation method involves mixing the membrane-forming lipids in an organic solvent, removal of the organic solvent to form a layer of the membrane-forming lipid, subsequent dispersion of the membrane-forming lipid in an aqueous solution, and further size reduction by mechanical treatment.

Alternatively, the preparation method involves mixing the membrane-forming lipids in an organic solvent, subsequent dispersion of the membrane-forming lipid containing phase in an aqueous solution, removal of the organic solvent, and further size reduction by mechanical treatment.

In an embodiment of the invention, the method for preparing a tooth whitening composition comprises:
1) the step of mixing at least one membrane-forming lipid in an organic solvent in a vessel, and
   either
2a) dispersing the mixture obtained in step 1) in an aqueous solution comprising a reducing agent, and
3a) removing the organic solvent to obtain a composition comprising liposomes comprising the solution of the reducing agent,

In an embodiment of the invention, the method for preparing a tooth whitening composition comprises:
1) the step of mixing at least one membrane-forming lipid in an organic solvent in a vessel,
2b) removing the organic solvent of the mixture obtained in step 1) to obtain a layer of the membrane-forming lipid on the inner surface of the vessel, and
3b) adding an aqueous solution comprising a reducing agent to the layer of step 2b) to obtain a composition comprising liposomes comprising the solution of the reducing agent.

The solution of the reducing agent comprises optionally a buffer system.

Usually, the membrane-forming lipid is dissolved or dispersed in the at least one organic solvent, preferably dissolved. The organic solvent is usually selected from the group comprising chloroform, dichloromethane, methanol, ethanol, and mixtures thereof. In a preferred embodiment the organic solvent is selected from chloroform and a combination of chloroform and methanol.

In a preferred embodiment, the method further comprises a step of treating mechanically the liposomes obtained in step 3a) or 3b) to reduce their size.

The mechanical treatment may be carried out by known methods, such as sonication, extrusion or homogenization. In a preferred embodiment, the size of liposomes is reduced by sonication, preferably in an ultrasound bath.

### Membrane-Forming Lipid

The membrane-forming lipid suitable to be used in the method of the invention comprises a phospholipid.

The phospholipid may be selected from a group consisting of a natural phospholipid, a synthetic phospholipid, and combinations thereof. Lecithin is one of the natural resources for phospholipid. Lecithin is a mixture found in egg yolk and soy. It comprises a number of phospholipids including phosphatidylcholine (PC), phosphatidylethanolamine (PE), and phosphatidylinositol (P1). Natural phospholipids also include, e.g. hydrogenated soy PC (HSPC), sphingomyelin, and phosphatidylglycerol (PG).

Synthetic phospholipids include, but are not limited to, derivatives of phosphocholine (for example, DDPC (1,2-didecanoyl-sn-glycero-3-phosphocholine), DLPC (1,2-dilauroyl-sn-glycero-3-phosphocholine), DMPC (1,2-dimyristoyl-sn-glycero-3-phosphocholine), DPPC (1,2-dipalmitoyl-sn-glycero-3-phosphocholine), DSPC (1,2-distearoyl-sn-glycero-3-phosphocholine), DOPC (1,2-dioleoyl-sn-glycero-3-phospho-choline), POPC (1-dalmitoyl-2-oleoyl-sn-glycero-3-phosphocholine), DEPC (1,2-dierucoyl-sn-glycero-3-phosphocholine)), derivatives of phosphoglycerol (for example, DMPG (1,2-dimyristoyl-sn-glycero-3-phosphorylglycerol), DPPG (1,2-dipalmitoyl-sn-glycero-3-phosphorylglycerol), DSPG (1,2-distearoyl-sn-glycero-3-phosphorylglycerol), POPG (1-palmitoyl-2-oleoyl-sn-glycero-3-phosphoglycerol)), derivatives of phosphatidic acid (for example, DMPA (1,2-dimyristoyl-sn-glycero-3-phosphate), DPPA (1,2-dipalmitoyl-sn-glycero-3-phosphate), DSPA (1,2-distearoyl-sn-glycero-3-phosphate)), derivatives of phosphoethanolamine (for example, DMPE (1,2-dimyristoyl-sn-glycero-3-phosphoethanolamine), DPPE (1,2-dipalmitoyl-sn-glycero-3-phosphoethanolamine), DSPE (1,2-distearoyl-sn-glycero-3-phosphoethanolamine), DOPE (1,2-dioleoyl-sn-glycero-3-phosphoethanolamine)), derivatives of phosphoserine (for example, DOPS (1,2-dioleoyl-sn-glycero-3-phosphoserine)), PEG derivatives of phospholipid (for example, PEG-phospholipid, polyglycerin-phospholipid, functionalized-phospholipid, and terminal activated-phospholipid), or salts thereof.

In one embodiment of the present invention, the phospholipid is a hydrogenated phospholipid, specifically, dipalmitoylphosphatidylcholine (DPPC).

The concentration of the at least one membrane-forming lipid in the composition comprising liposomes obtained in step 3a) or step 3b) is usually comprised between 1 mM and 100 mM, preferably between 5 mM and 50 mM, more preferably between 10 mM and 40 mM, yet more preferably between 15 mM and 30 mM, and yet more preferably between 15 mM and 25 mM. In a preferred embodiment, the concentration is 20 mM.

The properties of the membrane may be modified by combining the phospholipid with further components. In an embodiment of the invention, the membrane-forming lipid comprises a phospholipid and a further component. Further components may be incorporated to the membrane as disclosed in, for example, He et al., Acta Pharm. Sinica B, 2019, 9(1), 36-48; Fonseca et al., Biochim. Biophys. Acta Biomembranes, 1996, 1279 (2), 259-265; and Milla et al., Current Drug Metabol., 2012, 13(1), 105-119. In the context of the invention, further components that may be incorporated to the membrane are, for example, cholesterol; stearylamine; soybean-derived sterols; bile salts, such as sodium glycocholate, sodium taurocholate and sodium deoxycholate; polymers, such as polysaccharides, collagen, chitosan, chitosan derivatives, such as N-trimethyl chitosan and methylated N-(4-N,N-dimethylaminobenzyl) chitosan, polyethylene glycols, polyethylene glycol derivatives, such as PEG-distearoylphosphatidylethanolamine; and non-ionic surfactants, such as Tween 80.

In a preferred embodiment, the membrane-forming lipid comprises a phospholipid and a further component selected from cholesterol, stearylamine, soybean-derived sterol, bile salt, polysaccharide, collagen, chitosan, chitosan derivatives, polyethylene glycol, polyethylene glycol derivatives, non-ionic surfactant, and mixtures thereof.

In a more preferred embodiment, the membrane-forming lipid is combined with cholesterol. The presence of cholesterol as a component of the membrane-forming lipid improves the fluidity of the bilayer membrane, reduces the permeability of water-soluble molecules through the membrane, and improves the stability of bilayer membrane in the presence of biological fluids.

### Reducing Agent

A reducing agent (also called a reductant or reducer) is an element or compound that loses (or “donates”) an electron to another chemical species in a redox chemical reaction.

In the context of the present invention, a reducing agent is selected from the group of sulphur containing compounds such as dithionite, metabisulfite, sulphite, bisulfite, and alkaline metal salts thereof. Preferably the reducing agent is selected from sodium dithionite, potassium dithionite, sodium metabisulfite, potassium metabisulfite, sodium sulfite, potassium sulfite, sodium bisulfite, and potassium bisulfite, and more preferably from sodium metabisulfite and potassium metabisulfite.

The concentration of the reducing agent in the aqueous solution of step 2a) or step 2b) of the method is usually comprised between 0.01 M to 0.5 M, preferably between 0.01 M and 0.4 M, more preferably between 0.05 M and 0.3 M, and yet more preferably between 0.1 M and 0.2 M. In one preferred embodiment, the concentration is 0.1 M.

The aqueous solution of reducing agent usually has a pH value comprised between 2 and 8, preferably between 3 and 7.5, more preferably between 4 and 7, yet more preferably between 5.5 and 6.5, and more preferably 6.0. In one preferred embodiment the aqueous solution has the pH resulting from the dissolution of the reducing agent in water. For example, a 0.47 M solution of sodium metabisulfite has a pH value of about 2.9.

### Buffer System

The aqueous solution comprising the reducing agent comprises, in a preferred embodiment, a buffer system.

In that embodiment, the aqueous solution of reducing agent comprises a buffer system to adjust the pH to a value comprised between 4 and 7, preferably between 5.5 and 6.5, and more preferably 6.0. In a preferred embodiment the aqueous solution of reducing agent is buffered to pH 5.5, and in another preferred embodiment to pH 6.5.

The buffer system is selected by the skilled person among known buffer systems. Preferably the buffer system comprises citric acid and disodium citrate. If necessary, pH value may be further adjusted by adding any acid, such as hydrochloric acid, or base, such as sodium hydroxide.

### Liposomes

Liposomes comprising the membrane-forming lipid and reducing agent are obtained in step 1) of the method of the invention. In a preferred embodiment, liposomes also comprise an aqueous solution comprising a buffer system.

As used in this description, “liposome” is used to describe oligo-lamellar lipid vesicles comprising one or more natural or synthetic lipid bi-layers surrounding an internal aqueous phase.

Liposomes are commonly used in cosmetic formulations for improving dermal penetration of actives. As is well known in the art, liposomes are spherical vesicles with sizes generally in the range between about 60 nm and 300 nm and are most often composed of phospholipids, such as phosphatidylcholine, which form at least one phospholipid bilayer, but may also include other lipids, such as egg phosphatidylethanolamine. Liposomes contain hydrophilic cores in which hydrophilic actives may be encapsulated, while hydrophobic actives are incorporated in the bilayer, so liposomes are suitable carriers for both hydrophilic and lipophilic actives (Knoth et al., Nanocarrier-Based Formulations: Production and Cosmeceutic Applications, in: Cosmetic Formulation. Principles and Practice, Benson H. A. E., Roberts M. S., Rodrigues Leite-Silva V. and Walters K. A., editors, CRC Press, 2019).

Liposomes usually are classified either by the method of their preparation or by the number of bilayers present in the vesicle, or by their size.

According to the method of the invention, the obtained liposomes usually show a particle size from 20 nm to 500 nm, and they are formed by a mixture of unilamellar vesicles and multilamellar vesicles. By high-pressure extrusion through, for example, very small pore polycarbonate it is possible to reduce the average diameter of the liposomes to about 60 nm-80 nm after several passes. It is known that when reducing the size of liposomes, they tend to become unilamellar.

### Tooth Whitening Composition

It is an aspect of the invention a tooth whitening composition obtainable according to the process of the invention.

The tooth whitening composition of the invention comprises an aqueous solution comprising a reducing agent, and liposomes, comprising at least one membrane-forming lipid and an aqueous solution comprising a reducing agent.

In a preferred embodiment, the tooth whitening composition consists essentially of an aqueous solution comprising a reducing agent, and liposomes, comprising at least one membrane-forming lipid and an aqueous solution comprising a reducing agent.

In a preferred embodiment the tooth whitening composition further comprises a buffer system.

In a more preferred embodiment, the tooth whitening composition consists essentially of an aqueous solution comprising a reducing agent, a buffer system, and liposomes comprising at least one membrane-forming lipid, and an aqueous solution comprising a reducing agent, and a buffer system.

Preferably the reducing agent is selected from the group of sulphur containing compounds such as metabisulfite, sulphite, bisulfite, and alkaline metal salts thereof. Preferably the reducing agent is selected from sodium metabisulfite, potassium metabisulfite, sodium sulfite, potassium sulfite, sodium bisulfite, and potassium bisulfite, and more preferably from sodium metabisulfite and potassium metabisulfite.

Preferably the membrane-forming lipid comprises a phospholipid, more preferably a hydrogenated phospholipid, and yet more preferably dipalmitoylphosphatidylcholine (DPPC).

Usually the tooth whitening composition comprises further flavouring agents, sweeteners, preservatives, stabilizers or mixtures thereof to improve the acceptance by the user.

### Use

It is another aspect of the invention the non-therapeutic cosmetic use of that composition for tooth whitening.

A cosmetic effect relates to beautify and/or improve the appearance of teeth. A cosmetic effect does not involve any therapeutic effect, i.e., cosmetics are not intended to prevent or ameliorate any disease. A cosmetic effect is, for example, the whitening of teeth.

The composition of the invention may be used for whitening vital teeth, non-vital teeth, and prosthesis.

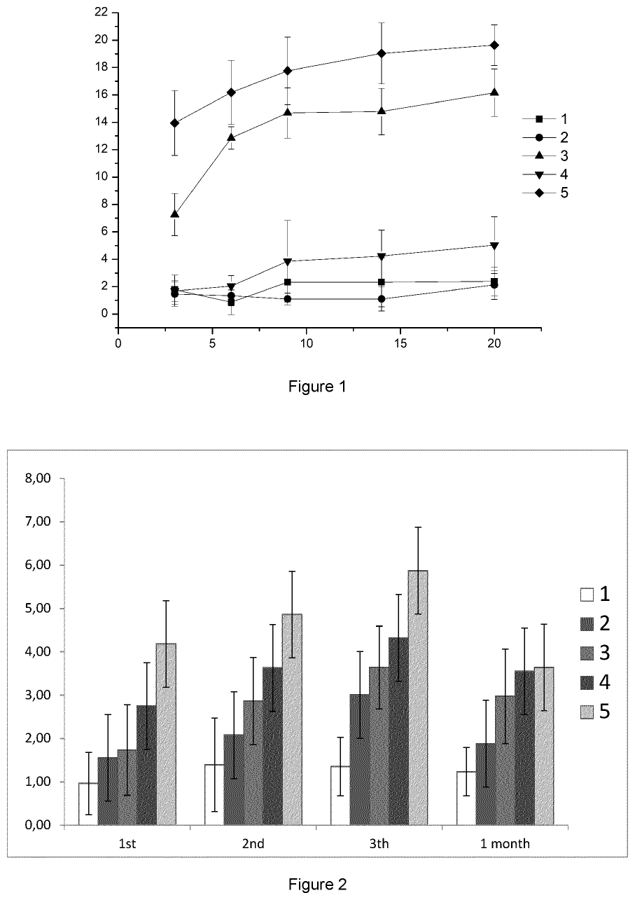

Tooth whitening trials with the composition of the invention show surprisingly an improved whitening effect, much higher than expected for the components alone as shown in FIG. 1. The composition of the invention comprising a reducing agent partially encapsulated in liposomes show a better result than that obtained using only the reducing agent, or only liposomes. It is observed that liposome encapsulation provides stability to the reducing agent and creates a layer on the enamel to favour penetration of the reducing agent to carry out the whitening activity.

Tooth whitening experiments may be carried out with bovine incisors or extracted human permanent maxillary central incisor, for example, as disclosed in Desai et al., op. cit. These specimens are usually cleaned of gross debris and the root cut using a diamond saw, and then may be preserved in 0.2% sodium azide solution until the experiment performance. The discolouration may be obtained by staining the tooth surface using a tannic acid solution. The whitening treatments may be carried out following a short laboratory treatment up to 20 minutes, performing colorimetric measurements at different times in order to monitor the whitening effect over time, or treatments according to ISO 28399 with longer treatments at 5-day intervals, preserving the tooth between times in artificial saliva, prepared according to the modified Shellis solution, as disclosed in R. P. Shellis, Effects of a Supersaturated Pulpal Fluid on the Formation of Caries-Like lesions on the Roots of Human Teeth, Caries Res., 1994, 28, 14-20. In both cases, the tooth whitening composition of the invention shows a fast and higher whitening performance in comparison to single components (reducing agent or liposomes) and to prior art treatments (hydrogen peroxide or carbamide peroxide).

Surprisingly, the composition of the invention is characterized by a fast and high whitening performance. The efficiency of the composition allows short treatment times and a reduction of secondary effects derived from the long application times of actual treatments.

The present invention comprises the following embodiments:
1. A method for preparing a tooth whitening composition, characterized in that it comprises:
   1) the step of mixing at least one membrane-forming lipid in an organic solvent in a vessel, and
      either
   2a) dispersing the mixture obtained in step 1) in an aqueous solution comprising a reducing agent, and
   3a) removing the organic solvent to obtain a composition comprising liposomes comprising the solution of the reducing agent,
      or
   2b) removing the organic solvent of the mixture obtained in step 1) to obtain a layer of the membrane-forming lipid on the inner surface of the vessel, and
   3b) adding an aqueous solution comprising a reducing agent to the layer of step 2b) to obtain a composition comprising liposomes comprising the solution of the reducing agent.
2. The method according to embodiment 1, characterized in that it comprises:
   1) the step of mixing at least one membrane-forming lipid in an organic solvent in a vessel,
   2a) dispersing the mixture obtained in step 1) in an aqueous solution comprising a reducing agent, and
   3a) removing the organic solvent to obtain a composition comprising liposomes comprising the solution of the reducing agent,
3. The method according to embodiment 1, characterized in that it comprises:
   1) the step of mixing at least one membrane-forming lipid in an organic solvent in a vessel,
   2b) removing the organic solvent of the mixture obtained in step 1) to obtain a layer of the membrane-forming lipid on the inner surface of the vessel, and
   3b) adding an aqueous solution comprising a reducing agent to the layer of step 2b) to obtain a composition comprising liposomes comprising the solution of the reducing agent.
4. The method according to any of embodiments 1 to 3, characterized in that the organic solvent is selected from the group comprising chloroform, dichloromethane, methanol, ethanol, and mixtures thereof.
5. The method according to embodiment 4, characterized in that the organic solvent is selected from chloroform and a combination of chloroform and methanol.
6. The method according to any one of embodiments 1 to 5, characterized in that it further comprises a step of treating mechanically the liposomes obtained in step 1) to reduce their size.
7. The method according to embodiment 6, characterized in that the step of mechanically treating the liposomes is carried out by sonication, extrusion or homogenization.
8. The method according to embodiment 7, characterized in that the step of mechanically treating the liposomes is carried out by sonication.
9. The method according to any one of embodiments 1 to 8, characterized in that the at least one membrane-forming lipid comprises a phospholipid.
10. The method according to embodiment 9, characterized in that the phospholipid is selected from the group consisting of a natural phospholipid, a synthetic phospholipid, and combinations thereof.
11. The method according to embodiment 10, characterized in that the natural phospholipid is selected from phosphatidylcholine (PC), phosphatidylethanolamine (PE), phosphatidylinositol (PI), hydrogenated soy PC (HSPC), sphingomyelin, phosphatidylglycerol (PG), and mixtures thereof.
12. The method according to embodiment 10, characterized in that the synthetic phospholipid is selected from derivatives of phosphocholine, derivatives of phosphoglycerol, derivatives of phosphatidic acid, derivatives of phosphoethanolamine, derivatives of phosphoserine, PEG derivatives of phospholipid, and mixtures thereof.
13. The method according to embodiment 12, characterized in that the synthetic phospholipid is selected from DDPC, DMPC, DPPC, DSPC, DOPC, POPC, DEPC, DMPG, DPPG, DSPG, POPG, DMPA, DPPA, DSPA, DMPE, DPPE, DSPE, DOPE, DOPS, and mixtures thereof.
14. The method according to embodiment 13, characterized in that the synthetic phospholipid is dipalmitoylphosphatidylcholine (DPPC).
15. The method according to any one of embodiments 1 to 14, characterized in that the concentration of the at least one membrane-forming lipid in the composition comprising liposomes obtained in step 3a) or step 3b) is comprised between 1 mM and 100 mM, preferably between 5 mM and 50 mM, more preferably between 10 mM and 40 mM, yet more preferably between 15 mM and 30 mM, yet more preferably between 15 mM and 25 mM and most preferably is 20 mM.
16. The method according to any one of embodiments 1 to 14, characterized in that the membrane-forming lipid comprises a phospholipid and a further component.
17. The method according to embodiment 16, characterized in that the further component is selected from cholesterol, stearylamine, soybean-derived sterol, bile salt, polysaccharide, collagen, chitosan, chitosan derivatives, polyethylene glycol, polyethylene glycol derivatives, non-ionic surfactant, and mixtures thereof.
18. The method according to embodiment 17, characterized in that the further component is cholesterol.
19. The method according to any one of embodiments 1 to 18, characterized in that the reducing agent is selected from the group of dithionite, metabisulfite, sulphite, bisulfite, and alkaline metal salts thereof.
20. The method according to embodiment 19, characterized in that the reducing agent is selected from sodium dithionite, potassium dithionite, sodium metabisulfite, potassium metabisulfite, sodium sulfite, potassium sulfite, sodium bisulfite, and potassium bisulfite, and more preferably from sodium metabisulfite and potassium metabisulfite.
21. The method according to embodiment 20, characterized in that the reducing agent is selected from sodium metabisulfite and potassium metabisulfite.
22. The method according to any one of embodiments 1 to 21, characterized in that the concentration of the reducing agent in the aqueous solution of step 2a) or step 2b) of the method is comprised between 0.01 M to 0.5 M, preferably between 0.01 M and 0.4 M, more preferably between 0.05 M and 0.3 M, yet more preferably between 0.1 M and 0.2 M, and most preferably is 0.1 M.
23. The method according to any one of embodiments 1 to 22, characterized in that the aqueous solution has the pH value comprised between 2 and 8, preferably between 3 and 7.5, more preferably between 4 and 7, yet more preferably between 5.5 and 6.5, and more preferably 6.0.
24. The method according to embodiment 23, characterized in that the aqueous solution has the pH resulting from the dissolution of the reducing agent in water.
25. The method according to any one of embodiments 1 to 23, characterized in that the aqueous solution of reducing agent comprises a buffer system.
26. The method according to embodiment 25, characterized in that the aqueous solution of reducing agent comprises a buffer system to adjust the pH to a value comprised between 4 and 7, yet more preferably between 5.5 and 6.5, and more preferably 6.0.
27. A tooth whitening composition obtainable according to the process of any one of embodiments 1 to 26.
28. The tooth whitening composition according to embodiment 27, characterized in that it comprises an aqueous solution comprising a reducing agent, and liposomes, comprising at least one membrane-forming lipid and an aqueous solution comprising a reducing agent.
29 The tooth whitening composition according to embodiment 28, characterized in that it further comprises a buffer system.
30. The tooth whitening composition according to embodiment 28, characterized in that it consists essentially of an aqueous solution comprising a reducing agent, and liposomes comprising at least one membrane-forming lipid and an aqueous solution comprising a reducing agent.
31. The tooth whitening composition according to embodiment 30, characterized in that it consists essentially of an aqueous solution comprising a reducing agent, a buffer system, and liposomes comprising at least one membrane-forming lipid, and an aqueous solution comprising a reducing agent and a buffer system.
32. The tooth whitening composition according to any of embodiments 27 to 31, characterized in that it further comprises flavouring agents, sweeteners, or mixtures thereof.
33. Non-therapeutic cosmetic use of composition according to any one of embodiments 27 to 32 for tooth whitening.
34. The use according to embodiment 33, characterized in that the composition is used for whitening vital teeth, non-vital teeth, and prosthesis.

In the following examples, particular embodiments of the composition of the invention are shown.

### EXAMPLES

### Colorimetric Measurements

For colour measurements, a contact type spectrophotometer Konica Minolta CR-321 Chroma Meter Colorimeter Bundled W/ DP-301 Data Processor, obtaining colour coordinates, L*, a*, and b*, was used.

The overall colour change is expressed as ΔE*ab, from the Commission Internationale de l'Eclairage, relative to the baseline colour parameters, and using the following equation:

ΔE*_{ab}=[(ΔLₐ*)2+(Δb*)²]^{1/2}

For 20 minutes whitening, measurements were performed at baseline, and after 3, 6, 9, 14 and 20 minutes of treatment.

For clinic-like whitening, measurements were performed at baseline (T1), 1-day post 1st application session (T2), 1-day post 2nd session (T3), 1-day post 3rd session (T4), and 1-month post whitening follow-up (T5).

The measurements were taken in the flat polished surface of the specimens.

### Nanoindentation Measurements

Enamel surface Young modulus of elasticity and hardness of two specimens of each treatment were determined by nanoindentation test using a Berkovich tip mounted on a nanoindenter (Nanoindenter XP-MTS). Before each test, the Berkovich diamond indenter was calibrated on a standard fused silica specimen, as disclosed in Nanoindentation—Ensuring Accuracy and Reliability Using Standard Specimens, Fischer-Cripps Labs., 2010 (https://www.azom.com/article.aspx?ArticleID=5415). Hardness is given by the equation below:

$$H = \frac{{P}_{max}}{{A}_{r}}$$

To convert the Vickers hardness number to SI units the hardness number in kilograms-force per square millimeter (kgf/mm²) has to be multiplied with the standard gravity (9.806 65) to get the hardness in MPa (N/mm²) and furthermore divided by 1000 to get the hardness in GPa.

### Preparative Example 1: Preparation of Specimens for Whitening Treatments

Bovine teeth (specimens) were cleaned of gross debris and the root was cut using a diamond saw, then preserved in 0.2 wt. % sodium azide solution until the experiment performance. The crowns were stained for 5 days using a tannic acid staining solution with a concentration of 80 g/L, at 37° C. and under stirring. The stained teeth were embedded in self-curing polyacrylic cylinders. The surface was polished to expose a window of 3 mm×3 mm enamel surface which will permit the surface roughness and colorimetric measurements. Specimens were ground using a sequence starting at P400 and sequentially increasing to P4000 silicon carbide paper under a constant flow of water. A diamond polishing suspension with a mean particle size of 1 μm followed by a slurry of aluminium oxide with a mean particle size of 0.3 μm were used for the final polishing. Finally, they were placed in artificial saliva, prepared according to the modified Shellis solution, and adjusted to pH 7.0 for 24 h prior to initiating the experiment.

The whitening treatments used in this description were performed under different conditions:
a) Tooth whitening treatments at 20 minutes according to 2² factorial design

The treatments were applied above the flattened surface of the specimens for a total of 20 minutes, performing colorimetric measurement at 3, 6, 9, 14 and 20 minutes in order to follow-up the kinetics of the whitening effect. To stop the whitening reaction during the colorimetry, the treatment material was removed from the specimens using an electric toothbrush while rinsing with miliQ water for 30 seconds.
b) Tooth whitening treatments at 20 minutes according to 2³ factorial design

The treatments were applied above the flattened surface of the specimens fora total of 20 minutes.
c) Whitening treatments according to ISO 28399

The treatment regimens were performed in three applications (1h each) at 5-day intervals for 3 repetitions, using 25 μL of the product in order to cover the exposed surface. The whitening solutions were replenished with 50 μL once during the 20-minute application session. The negative (NC) and positive control (PC) groups will be treated according to the International Organization for Standardization (ISO) 28399 protocol with miliQ water and 1.0 wt. % citric acid adjusted to pH 3.9 for 60 minutes, respectively. In all cases, after each treatment repetition teeth will be stored in artificial saliva.

Treatments carried out on the above disclosed specimens are able to provide relative values (i.e. provision of comparative results within the experiments set), but they do not provide reproducible absolute values, because the result depends on the staining process.

### Preparative Example 2: Preparation of Hydrogen Peroxide Solution

A 35 wt. % H₂O₂ (HP) aqueous solution was adjusted to pH 8.0 with NaOH. That solution was freshly prepared immediately before its use.

### Preparative Example 3: Preparation of Carbamide Peroxide Solution

A 16 wt. % carbamide peroxide (CP) aqueous solution was adjusted to pH 8.0 with NaOH. The solution was freshly prepared immediately before its use.

### Preparative Example 4: Preparation of Sodium Metabisulfite Solution for 2² Factorial Design Tooth Whitening Treatments

A solution of sodium metabisulfite 0.47 M was prepared by dissolving 8.9 g of sodium metabisulfite in 100 mL of deionized water. The pH value of this solution was 2.9.

### Preparative Example 5: Preparation of Sodium Metabisulfite Solution for 2³ Factorial Design Tooth Whitening Treatments

Solutions of sodium metabisulfite were prepared in a citric/citrate buffer solution adjusted to pH 5.5 or 6.5, according to the following procedure:
1) Preparing a 0.5 M citric acid solution (CAS)
2) Preparing a 0.5 M sodium citrate solution (SCS)
3) Mixing 7.2 mL of CAS and 42.8 mL of SCS
4) Using the solution in step 3 to solubilize the specific amount of sodium metabisulfite for the tooth whitening test
5) Adjusting pH with NaOH 3M to the desired pH (between 5.5 or 6.5)
6) Adjusting the final volume to 100 mL using deionized water.

For preparing a 0.1 M sodium metabisulfite solution, 1.9 g of sodium metabisulfite was weighed and solubilized in a mixture of 7.2 mL of CAS and 42.8 mL of SCS. The pH value was adjusted to 5.5 using 3M NaOH. The solution was adjusted to 100 mL with deionized water.

### Preparative Example 6: Preparation of DPPC Liposomes

A 20 mM liposomal solution was prepared according to the following procedure:
1) 0.4 mL of a 100 mM DPPC chloroform solution were pipetted into a round rotary flask.
2) A thin layer was created in the flask, by evaporating all the chloroform while applying rotation to the flask, using a rotary evaporator.
3) 2 mL of water was added to the flask, and then mixed for 15 minutes with an ultrasound bath.

The 20 mM liposomal solution was freshly prepared before the application and stored at 4° C. until its use.

### Example 1: Preparation of the Tooth Whitening Composition (Sodium Metabisulfite 0.47 M Partially Encapsulated in Liposomes)

A 20 mM liposomal solution of sodium metabisulfite 0.47 M was prepared according to the following procedure:
1) 0.4 mL of a 100 mM DPPC chloroform solution were pipetted into a round rotary flask.
2) A thin layer was created in the flask, by evaporating all the chloroform while applying rotation to the flask, using a rotary evaporator.
3) 2 mL of a 0.47 M sodium metabisulfite solution prepared according to the Preparative example 4, was added to the flask, and then mixed for 15 minutes with an ultrasound bath.

The 20 mM liposomal solution of sodium metabisulfite 0.47 M was freshly prepared before the application and stored at 4° C. until its use. pH value was 2.9, and the concentration of DPPC was 20 mM.

### Example 2: Preparation of the Tooth Whitening Composition (Sodium Metabisulfite 0.1 M Partially Encapsulated in Liposomes)

A 20 mM liposomal solution of sodium metabisulfite 0.1 M was prepared according to the following procedure:
1) 0.4 mL of a 100 mM DPPC chloroform solution were pipetted into a round rotary flask.
2) A thin layer was created in the flask, by evaporating all the chloroform while applying rotation to the flask, using a rotary evaporator.
3) 2 mL of a 0.1 M sodium metabisulfite solution prepared according to the Preparative example 4, was added to the flask, and then mixed for 15 minutes with an ultrasound bath.

The 20 mM liposomal solution of sodium metabisulfite 0.1 M was freshly prepared before the application and stored at 4° C. until its use. pH value was 6.0, and the concentration of DPPC was 20 mM.

### Example 3: Tooth Whitening Treatments According to 2² Factorial Design

Tooth whitening trials were carried out with a composition of the invention (Example 1) and three comparative compositions, according to a 2² factorial experimental design, which is suitable to quantify the effect of the components and interactions between the factors, i.e. synergistic effect.

Trials were carried out on the specimens following a treatment of 20 minutes, as disclosed in Preparative example 1.

Factors and levels are shown in Table I:

TABLE I

| Factor | Low level | High level |
|---|---|---|
| Sodium metabisulfite (M) | 0 | 0.47 |
| DPPC (mM) | 0 | 20.0 |

The response was the increase in the whiteness of the tooth, ΔE, measured as explainer earlier in this Examples section. The experiments were carried out in 8 specimens for each treatment.

Table II shows the experiments:

TABLE II

| Example | MBS | DPPC |
|---|---|---|
| Comparative 1 (control) | 0 | 0 |
| Comparative 2 (only MBS) (Preparative example 4) | 0.47 | 0 |
| Comparative 3 (only liposomes) (Preparative example 6) | 0 | 20 |
| Example 1 (invention) | 0.47 | 20 |

The responses were measured at different times. In Table III are shown the results for the increase in the whiteness of the tooth, ΔE±standard deviation, at 3, 6, 9, 14 and 20 minutes:

TABLE III

| Example | 3′ | 6′ | 9′ | 14′ | 20′ |
|---|---|---|---|---|---|
| Comparative 1 (control) | 1.8 ± 1.1 | 0.9 ± 0.9 | 2.3 ± 1.4 | 2.3 ± 1.8 | 2.4 ± 1.0 |
| Comparative 2 (only MBS) | 7.3 ± 1.5 | 12.9 ± 0.8 | 14.7 ± 1.8 | 14.8 ± 1.7 | 16.2 ± 1.7 |
| Comparative 3 (only liposomes) | 1.5 ± 0.9 | 1.3 ± 0,7 | 1.1 ± 0.4 | 1.1 ± 0.9 | 2.1 ± 1.0 |
| Example 1 (invention) | 14.0 ± 2.4 | 16.2 ± 2.3 | 17.8 ± 2.5 | 19.0 ± 2.2 | 19.6 ± 2.1 |

In FIG. 1 can be seen the kinetic of the tooth whitening for these four treatments and in comparison with the treatment using the composition of Preparative example 3, a 16 wt. % aqueous solution carbamide peroxide.

It can be clearly observed that the composition of the invention shows a synergistic effect, in particular at short times. The whiteness' increase of the composition of the invention (Example 1) at 3 minutes was 14, whereas the effect of MBS alone is only 7.3, and the liposomes alone did not produce any whitening effect.

Further, the whitening effect is faster than that of the standard prior art product, 16% aqueous solution of carbamide peroxide. Surprisingly, it was observed that at 3 minutes the whitening effect was substantially similar to that obtained at the end of the experiment, i.e. 20 minutes, showing a fast and high whitening performance, as shown in FIG. 1. The efficiency of the composition of the invention allows short treatment times and a significant reduction of secondary effects.

### Examples 4-11: Tooth Whitening Treatments According to a 2³ Factorial Design

Tooth whitening trials were carried out according to a 2³ factorial experimental design on the specimens disclosed in Preparative example 1, and following a treatment of 20 minutes, as disclosed earlier. Compositions were prepared following substantially the method disclosed in Example 2 (adapting concentrations of MBS and DPPC, as well as pH value). The experiments were carried out in triplicate.

Factors and levels are shown in Table IV:

TABLE IV

| Factor | Low level | High level |
|---|---|---|
| Sodium metabisulfite (M) | 0.01 | 0.1 |
| pH | 5.5 | 6.5 |
| DPPC (mM) | 2.0 | 20.0 |

Responses were the increase in the whiteness of the tooth, ΔE, and the enamel surface hardness of the tooth, expressed in GPa, both parameters measured after 20 minutes of treatment, as explained earlier in this Examples section.

Table V shows the treatments and the results:

TABLE V

| | | | | | Hardness |
|---|---|---|---|---|---|
| Example | MBS | pH | DPPC | ΔE | (GPa) |
| 4 | 0.01 | 5.5 | 2.0 | 4.0 ± 1.1 | 3.5 ± 1.2 |
| 5 | 0.1 | 5.5 | 2.0 | 6.3 ± 1.1 | 2.8 ± 0.3 |
| 6 | 0.01 | 6.5 | 2.0 | 4.4 ± 0.6 | 4.0 ± 1.2 |
| 7 | 0.1 | 6.5 | 2.0 | 3.5 ± 0.9 | 2.9 ± 0.3 |
| 8 | 0.01 | 5.5 | 20.0 | 5.7 ± 1.1 | 4.0 ± 0.7 |
| 9 | 0.1 | 5.5 | 20.0 | 16.2 ± 2.4 | 2.4 ± 0.9 |
| 10 | 0.01 | 6.5 | 20.0 | 7.1 ± 2.3 | 4.1 ± 0.2 |
| 11 | 0.1 | 6.5 | 20.0 | 3.8 ± 0.6 | 3.3 ± 0.2 |

The responses were measured at 20 minutes. It was observed an increase of whiteness of the tooth in all treatments.

Modelling the response from these results, an enhanced formula was obtained, as described in example 2.

### Example 12: Tooth Whitening Treatments According to ISO 28399

Tooth whitening treatments according to ISO 28399 were carried out as disclosed in Preparative example 1.

The tested products were:

1) Negative control (water)
2) Citric acid aqueous solution (1 wt. %, pH 3.9), positive control
3) 16 wt. % carbamide peroxide solution (Preparative example 3)
4) 35 wt. % hydrogen peroxide solution (Preparative example 2)
5) 0.1 M sodium metabisulfite liposome solution (Example 2)

FIG. 2 shows the results obtained regarding the increase in the whiteness for those products, represented as ΔE at 20 minutes of treatment. The effectiveness of the product of the invention is better than the prior art treatments (carbamide peroxide and hydrogen peroxide) after the first and second treatment, and comparable to HP and better than CP after the third treatment and after one month of follow up.

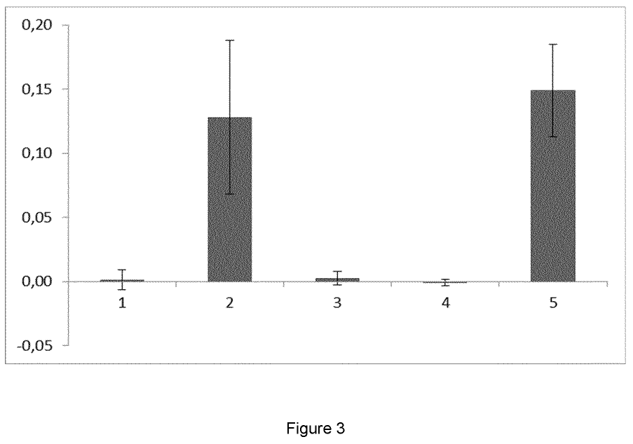

The roughness of the teeth after the treatments was measured according to ISO 28399. The increase in roughness is represented as ΔRa in FIG. 3 for each one of the treatments. It can be observed that the composition according to the invention showed an effect comparable to that of citric acid, used as positive control, being consequently suitable for dental use.

1. A method for preparing a tooth whitening composition, characterized in that it comprises:
1) the step of mixing at least one membrane-forming lipid in an organic solvent in a vessel, and
either
2a) dispersing the mixture obtained in step 1) in an aqueous solution comprising a reducing agent, and
3a) removing the organic solvent to obtain a composition comprising liposomes comprising the solution of the reducing agent,
or
2b) removing the organic solvent of the mixture obtained in step 1) to obtain a layer of the membrane-forming lipid on the inner surface of the vessel, and
3b) adding an aqueous solution comprising a reducing agent to the layer of step 2b) to obtain a composition comprising liposomes comprising the solution of the reducing agent.

2. The method according to claim 1, characterized in that the organic solvent is selected from the group comprising chloroform, dichloromethane, methanol, ethanol, and mixtures thereof.

3. The method according to claim 1 or 2, characterized in that it further comprises a step of treating mechanically the liposomes obtained in step 1) to reduce their size.

4. The method according to any one of claims 1 to 3, characterized in that the at least one membrane-forming lipid comprises a phospholipid.

5. The method according to claim 4, characterized in that the phospholipid is selected from the group consisting of a natural phospholipid, a synthetic phospholipid, and combinations thereof.

6. The method according to claim 5, characterized in that the natural phospholipid is selected from phosphatidylcholine (PC), phosphatidylethanolamine (PE), phosphatidylinositol (PI), hydrogenated soy PC (HSPC), sphingomyelin, phosphatidylglycerol (PG), and mixtures thereof.

7. The method according to claim 5, characterized in that the synthetic phospholipid is selected from derivatives of phosphocholine, derivatives of phosphoglycerol, derivatives of phosphatidic acid, derivatives of phosphoethanolamine, derivatives of phosphoserine, PEG derivatives of phospholipid, and mixtures thereof.

8. The method according to any one of claims 1 to 7, characterized in that the concentration of the at least one membrane-forming lipid in the composition comprising liposomes obtained in step 3a) or step 3b) is comprised between 1 mM and 100 mM.

9. The method according to any one of claims 1 to 8, characterized in that the reducing agent is selected from the group of dithionite, metabisulfite, sulphite, bisulfite, and alkaline metal salts thereof.

10. The method according to any one of claims 1 to 9, characterized in that the concentration of the reducing agent in the aqueous solution of step 2a) or step 2b) of the method is comprised between 0.01 M to 0.5 M.

11. The method according to any one of claims 1 to 10, characterized in that the aqueous solution has the pH value comprised between 2 and 8.

12. The method according to any one of claims 1 to 11, characterized in that the aqueous solution of reducing agent comprises a buffer system.

13. A tooth whitening composition obtainable according to the process of any one of claims 1 to 12.

14. The tooth whitening composition according to claim 13, characterized in that it comprises an aqueous solution comprising a reducing agent, and liposomes, comprising at least one membrane-forming lipid and an aqueous solution comprising a reducing agent.

15. Non-therapeutic cosmetic use of composition according to claim 13 or 14 for tooth whitening.
